(19) United States
(12) Patent Application Publication
(10) Pub. No.: US 2016/0354112 A1
(43) Pub. Date: Dec. 8, 2016

(54) NEEDLE GUIDE AND MEDICAL INTERVENTION SYSTEM

(71) Applicant: KONINKLIJKE PHILIPS N.V., Eindhoven (NL)

(72) Inventors: JACEK LUKASZ KUSTRA, EINDHOVEN (NL); GERNOT JOSEPH PIETER MARIE EGGEN, OOSTERHOUT (NL); GUILLAUME LEOPOID THEODORUS FREDERIK HAUTVAST, EINDHOVEN (NL)

(21) Appl. No.: 15/116,358

(22) PCT Filed: Feb. 13, 2015
(86) PCT No.: PCT/EP2015/053059

§ 371 Date: Aug. 3, 2016

(30) Foreign Application Priority Data
Feb. 25, 2014 (EP) 14156553.1

(51) Int. Cl. A61B 17/34; A61B 34/20

(52) U.S. Cl. CPC A61B 17/3403; A61B 34/20; A61B 2017/3411; A61B 2034/2055

(57) ABSTRACT

The present invention relates to a medical intervention system which increases the safety of the patient or animal during a medical intervention comprising a needle insertion step. This object is achieved by a medical intervention system as claimed in claim for use during a medical intervention, comprising: a needle guide configured to guide needle insertion into a body for a medical intervention wherein the needle guide comprises restriction means for regulating the needle insertion resistance wherein the needle guide comprises an actuator configured for operating the restriction means in dependence of an input control signal and a medical data unit configured for storing and/or acquiring medical data; a control system, configured for receiving data from the medical data unit further configured to generate the input control signal for the restriction means based on the medical data in order to reduce the risk of penetration of a vulnerable area by the needle.

### FIELD OF THE INVENTION

The invention relates to a needle guide configured to guide needle insertion into a body for a medical intervention and medical intervention system comprising such needle guide.

### BACKGROUND OF THE INVENTION

U.S. Pat. No. 8,262,577 describes a method wherein needles are deployed in tissue under direct ultrasonic or other imaging. To aid in deploying the needle, a visual needle guide is projected on to the image prior to needle deployment. Once the needle guide is properly aligned, the needle can be deployed. After needle deployment, a safety boundary and treatment region is projected on to the screen. After confirming that the safety boundary and treatment regions are sufficient, the patient can be treated using the needle.

WO 2013/090528A1 discloses a method for controlling electric fields created by a plurality of electrodes. WO 2013/090528A1 furthermore shows a electrode guide that includes a plurality of electrode templates and an adjustable template securing apparatus. The templates may include apertures suitably sized and shape to form a friction fit with an electrode. Also the templates may include a friction plate operable to selectively change a friction force applied to one or more electrodes received by the template.

US2012/0203095A1 describes an intervention apparatus having a probe with an orifice insertion portion, the insertion portion being configured for insertion into an orifice of a patient. The apparatus also having an intervention tool securement and adjustment mechanism removably attached to the probe.

### SUMMARY OF THE INVENTION

It is an object of the invention to provide a medical intervention system which increases the safety of the patient or animal during a medical intervention comprising a needle insertion step.

This object is achieved by

a medical intervention system for use during a medical intervention, comprising:

a needle guide configured to guide needle insertion into a body for a medical intervention wherein the needle guide comprises restriction means for regulating the needle insertion resistance wherein the needle guide comprises an actuator configured for operating the restriction means in dependence of an input control signal and a- a medical data unit configured for storing and/or acquiring medical data;

a control system, configured for receiving data from the medical data unit further configured to generate the input control signal for the restriction means based on the medical data in order to reduce the risk of penetration of a vulnerable area by the needle.

Traditional therapy delivery systems rely on a needle guide or template grid which a user uses to guide the needles in two dimensions. The needles are then pushed until they reach a predefined position. Real-time imaging systems, such as ultrasound (2D/3D), are commonly used to provide visual feedback of the instrument insertion. Visualization systems used with these traditional therapy delivery devices have some limitation, i.e. usually a single slice is visualized at a time, providing limited feedback of the full extent of the needle extension. Furthermore, given existing time constrains in the therapy delivery and the amount of insertions in procedures such as brachytherapy (20+) or biopsies (12), several critical body structures can be mistakenly punctured leading to (severe) side effects. A body structure could an organ or part of an organ.

The needle guides or template grids used in current systems are simple passive grids, meaning that they are simply objects with a punctured grid for needle insertion and position guidance. Puncturing a grid hole that is not to be punctured does not result in any alarm or haptic feedback, while potentially causing damage to vulnerable body structures. Therefore embodiments of the invention propose an active needle guide, which regulates the needle insertion resistance. To this end the needle guide comprises restriction means. By regulation of the needle insertion resistance the chance of puncturing of vulnerable body structures can be decreased, which in turn increases the safety of the patient or animal during the medical intervention.

The restriction means could be a manual mechanism on the template that enables the user to close/lock one or more orifices. Also, the restriction means could be configured for actively regulating the needle insertion resistance in dependence of a input control signal. According to a further aspect of the invention, the needle guide further comprises an actuator configured for operating the restriction in dependence of an input control signal. The actuator could be detachably attachable to the needle guide. This is advantageous, because in this way one actuator can be used for different needle guides. The proposed needle guide's basic functionality mimics the current needle guide in the sense that the same needles are used in a similar setting as currently. However, as soon as a vulnerable body structure is about to be punctured, the needle actively acts on the needle insertion, by restricting, stopping or blocking (further) needle insertion. This increases the safety of the patient or animal during a medical intervention comprising a needle insertion step. The resistance to insertion can be based on either predetermined positions or updated in real-time through imaging.

A further embodiment of the needle guide is equipped with a position sensor or marker configured to be used for determination of the position of the needle guide with respect to a predetermined part of the body. In this embodiment, erroneous needle insertion can also be stopped, even when the needle guide has moved relative to the patient.

The restriction means for actively regulating the needle insertion resistance could for example be a brake, e.g. an electromagnetic brake or mechanical brake.

A further embodiment of the needle guide is also configured to provide an audio and/or visual signal to the user if a needle is within a certain distance from a vulnerable area. The audio and/or visual signal can be provided to the user before restricting, stopping or blocking of the needle takes place. This may help the user to correct the path of needle insertion at an earlier stage. Furthermore, in this way an additional way of warning the user is achieved, which will make it clearer to the user that the needle is within a certain distance from a vulnerable area.

Also, the audio and/or visual signal can be provided to the user at the same time or after restricting, stopping or blocking of the needle has taken place. In this way an additional way of warning the user is achieved, which will make it clearer to the user that the needle is within a certain margin from a vulnerable area.

The needle guide could be a grid comprising several orifices arranged to assist needle guidance during a medical intervention or it could be a needle guide comprising only a single orifice, which could be used to guide the needle to a predetermined position.

According to one aspect of the invention the needle guide could be part of a medical intervention system. One or more elements of the medical intervention system could be configured for providing the needle guide with input and/or receiving output from the needle guide.

According to another embodiment of the invention, the needle guide could be combined with a medical data unit and a control system. Medical data stored and/or acquired by the medical data unit could be provided to the control system. Based on the medical data, the control system could generate the input control signal for the needle guide.

When the needle guide is a grid template, the medical data could be information on what orifices should and/or should not be used during the medical intervention. The control system could send an input control signal to the orifices that should not be used as to increase the needle insertion resistance for these orifices. One of the advantages of this is that in this way can be prevented that by accident, a needle will be inserted in a wrong orifice.

Furthermore, in medical interventions comprising multiple needle insertions (like e.g. brachytherapy), the medical data may comprise information on a current grid orifice to be used for needle insertion and thereby also information on which grid orifice or grid orifices should not be currently used. Additionally, the grid template will open just one grid orifice at the time as part of a sequence that is provided by the control system. Similarly, the medical intervention system according to this embodiment is also useful to prevent accidental, unnecessary movement of needles, in order to reduce the amount of injury due to intervention itself E.g. in situations where there is one specific needle to be moved, while 20 needles are placed, it may be beneficial to lock the remaining 19 needles.

According to another embodiment of the invention, the medical data unit comprises a medical imaging system (e.g. 2D or 3D ultrasound, X-ray system, CT system, MRI system), an image segmentation unit, configured for segmentation of a body structure in the image and means for tracking a needle position (e.g. by means of the imaging system, EM tracking, optical shape sensing, a camera) , configured for tracking a position of the needle. According to this aspect, the control system is configured to generate the input control signal for the restriction means based on a distance between the segmented body structure and the needle position. This embodiment is advantageous, because in this way the restriction means could be controlled based on a current situation instead of the situation at the time an intervention plan was made. This embodiment is especially advantageous if the medical intervention system is configured to track the vulnerable body structure over time. This is especially advantageous when dealing with a body structure that moves or changes shape during the medical intervention. By tracking moving body structures unwanted puncturing can be prevented.

According to another embodiment of the invention, restriction means is configured for blocking, stopping or restricting needle insertion, when the needle tracking fails. In this way safety can be further increased, because moving a needle becomes more difficult or impossible as long as the information regarding the needle position is insufficient. According to another aspect of the invention, the restriction means is configured for blocking, stopping or restricting needle insertion, when tracking of the body structure fails.

According to another embodiment of the invention the information flow between the needle guide and the control system is reversed. The medical intervention system comprises a display. A manual mechanism on the template enables a user to close/lock one or more orifices. The control system will respond by sending the information to the display for displaying areas in the medical images that can or cannot be reached by a needle inserted through the needle guide. Also this aspect contributes to the safety of the patient or animal as showing which areas can and/or cannot be reached helps in the prevention of puncturing vulnerable body structures.

These and other aspects of the invention will be apparent from and elucidated with reference to the embodiments described hereinafter.

### BRIEF DESCRIPTION OF THE FIGURES

FIG. 1 illustrates a conventional needle guide.

FIG. 2 illustrates a needle guide according to embodiments of the invention.

FIG. 3 illustrates a restriction means according to embodiments of the invention

FIG. 4 illustrates a medical intervention system according to embodiments of the invention

### DETAILED DESCRIPTION OF THE INVENTION

In the context of a typical clinical scenario, a medical intervention pre-plan is performed based on pre-imaging data, wherein therapy delivery targets or biopsy targets are defined, together with on or more vulnerable area to avoid. An intervention goal is to deliver the intervention as close as possible to the pre-plan.

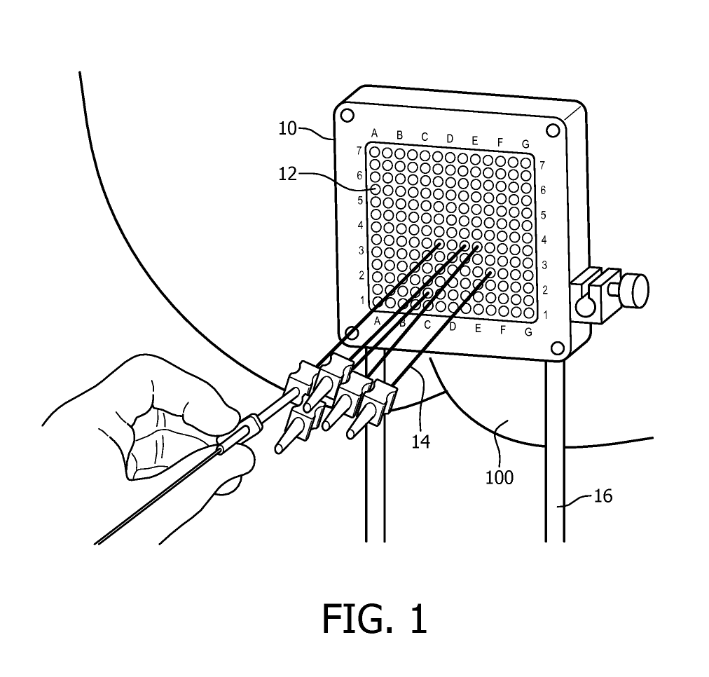

FIG. 1 illustrates a conventional needle guide 10. The needle guide shown in FIG. 1 comprises several orifices 12 to assist needle 14 insertion during a medical intervention. Positioning means 16 are used to position the needle guide correctly relative to the patient 100. The positioning means could be connectable to the patient table. After the patient and needle guide are positioned, the position of the needle guide can be calibrated with a medical imaging system 301. After this, based on a prescribed dose distribution an intervention plan (e.g. a HDR/LDR brachy treatment plan) can be calculated, which plan provides needle insertion positions required to deliver a dose distribution that matches the prescribed dose distribution within an acceptable range. Also a plan can be calculated on how the needle guide can be used to guide the needle to a predetermined location in order to perform a biopsy.

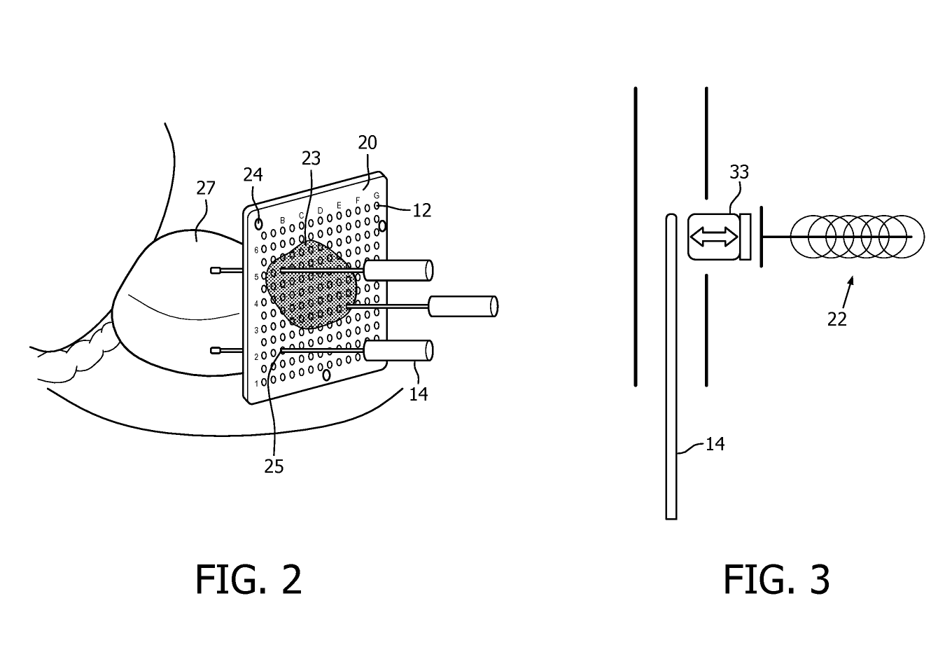

FIG. 2 illustrates an embodiment of a needle guide 20 according to the invention. The needle guide comprises one or more orifices 12 to assist in needle guidance. The needle guide comprises restriction means 33 (FIG. 3) for regulating the needle insertion resistance. In FIG. 2, area 23 is a projection of target 27 on the needle guide. The needle restriction means allow (further) needle insertion through the orifices 12 located in area 23. However, needle insertion is restricted or impossible through orifices outside area 23 because of increased insertion resistance in this orifice, e.g. further needle insertion through 25 is not possible. The restriction means could be operated manually or in dependence of a control input signal.

FIG. 3 illustrates an embodiment of restriction means. Brake 33 could be electrically controlled via an actuator, which in FIG. 3 is a coil 22. When electricity is applied to coil 22 magnetic flux attracts the brake, which will open the orifice. In other embodiments, the orifice can be closed by application of electricity to coil 22.

According to embodiments of the invention the needle guide comprises an actuator 22 configured for operating the restriction in dependence of an input control signal. The actuator 22 could be detachably attachable in such a way that one actuator could be used for different needle guides. A possible mechanical implementation of the brakes can be made by internally equipping the grid with electromagnetic brakes. In this case, several orifices have a friction plate which is triggered by an external electric signal. Variations of the implementation can be based on multiple restriction means (one per orifice) or a single actuator 22 which activates multiple restriction means on several orifices or rows and/or columns of orifices.

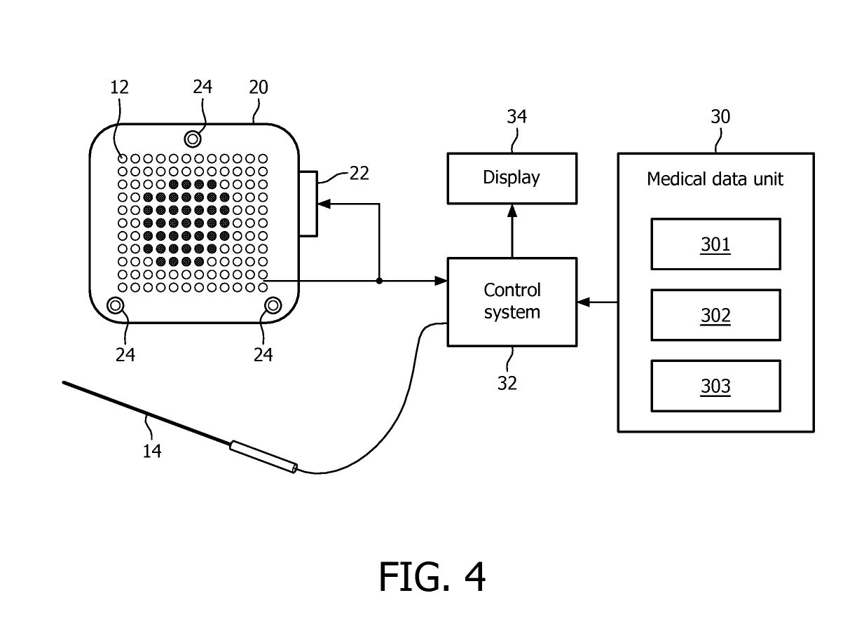

According to embodiments of the invention, the needle guide comprises a position sensor or marker 24 (FIG. 2, FIG. 4) configured for using for determination of the position of the needle guide with respect to predetermined part of the body 27. Examples of position sensors or markers are coils or markers visible to a camera, like e.g. LED. Also needles could be equipped with position sensors or markers, which could also be coils or fibers in case of optical shape sensing.

FIG. 4 illustrates a medical intervention system according to embodiments of the invention. The system comprises the needle guide as shown in FIG. 2, 20. Furthermore, the medical intervention system comprises a medical data unit 30 configured for storing and/or acquiring medical data. Furthermore, the medical intervention system comprises a control system 32, configured for receiving data from the medical data unit 30 further configured to generate the input control signal for the restriction means based on the medical data in order to reduce the risk of penetration of a vulnerable area by the needle.

The intervention plan may comprise information on which orifices are supposed to be used during the medical intervention for needle insertion and can be stored in the medical data unit 30. Possibly also the order in which the orifices are supposed to be used is stored in the medical data unit 30. During the medical intervention the control system can send the control input signal to the needle guide as to increase the needle insertion resistance for the orifices not to be used during the intervention. Also the control system can send the control input signal to the needle guide as to increase the needle insertion resistance for the orifices not to be used at a certain moment during the medical intervention. Keeping track of which needles have been successfully inserted could be done by allowing a user to provide the medical intervention system with input that a needle has been successfully inserted. Also, the medical intervention system could automatically detect if a needle has been successfully inserted by means of the intervention plan and needle tracking, possibly in combination with body structure tracking.

To make sure that the correct orifices are used during the medical intervention, the relative position of the needle guide to a predetermined part of the body needs to be known. The position can be determined by means of the position sensor or marker 24 or by means of a calibration process as described above.

According to a further embodiment of the invention, the medical data unit 30 comprises a medical imaging system, configured for image acquisition 301, an image segmentation unit 302, configured for segmentation of a body structure in the image and means 303 for tracking a needle position, configured for tracking a position of the needle. The medical imaging system could for example be a 2D or 3D ultrasound, an X-ray system, a CT system or an MRI system. Image segmentation can be performed manually, semi-automatically or automatically. Image segmentation methods are well known in the art of image processing and will therefore not be further discussed here. The means for tracking a needle position could for example be the medical imaging system, an EM tracking system, optical shape sensing

In order to avoid hitting and damaging vulnerable structures, such as the bladder during a medical intervention the medical imaging system is used to assist a user with needle guidance through the patient or animal body. In the case of using a 2D ultrasound scanner, this is usually placed at the bladder plane and a continuous observation of the imaging system is required to detect the needle passing through the imaging plane. To overcome this limitation, a 3D ultrasound scanner can be used, providing a full imaging of the intervention area. However, the direct visualization of 3D data in real time is far from trivial, which can lead to mistakes in the procedure. These mistakes can be overcome by presenting an augmented imaging, where segmentation data is overlaid on the original imaging together with the needle position, inferred from for example the image segmentation or electromagnetic (EM) tracking

The intervention goal is to deliver the intervention (e.g. therapy or taking of a biopsy) as close as possible to the pre-plan and/or intervention plan. Due to several changes occurring in the target 27 organs during the intervention, an organ tracking system can actively prevent the user from reaching vulnerable areas which are displaced and/or deformed. In this setting, the medical imaging system provides (real time) updated images during the medical intervention. Body structures can be tracked over time for example by using the initial segmentation in combination with image registration, also anatomical landmarks in combination with image, feature or pattern recognition can be used for body structure tracking Other methods are possible. Since, tracking methods are well known in the art of image processing, these methods will not be discussed in further detail. The means for tracking a needle position provides the relative needle position to the imaging data and the needle guide provides a guide to the needle insertions. The control system uses the imaging data and the means for tracking a needle position to define an appropriate needle guide response, and in case the needle is approaching a predefined body structure, the needle insertion resistance is increased as to restrict or prevent further needle insertion.

A position tracker for the needle is not an essential part of this system. Needle insertion may also be blocked, stopped or restricted based image information only, i.e. based on segmentations of the anatomy and the needle.

According to other embodiments of the system according to the invention the needle guide may also be used in a safety mechanism, preventing any needle insertion/movement in situations when this is recommendable. For example, this could be valuable if body structure tracking or needle tracking is temporarily unavailable.

According to further embodiments of the invention, the needle guide comprises an audio and/or visual indicator, configured for providing an audio and/or visual signal based on the input control signal. Also the medical intervention system may comprise the audio and/or visual indicator.

According to embodiments of the system according to the invention the information flow between the needle guide and the control system is reversed. In these embodiments the medical intervention system comprises a display 34. A manual mechanism on the template enables the user to close/lock on or more orifices. The control system will respond by determining what regions can be and/or cannot be reached from the one or more open orifices and send this information to the display 34. The display is configured for displaying the regions that can be and/or cannot be reached from the open orifices. This information could be combined with images acquired with the medical imaging system during the medical intervention. When the image segmentation unit detects patient movement, the locking of already placed needles should be released, to prevent unnecessary damage.

Whilst the invention has been illustrated and described in detail in the drawings and foregoing description, such illustrations and description are to be considered illustrative or exemplary and not restrictive; the invention is not limited to the disclosed embodiments and can be used for needle guidance various applications within the field medical interventions.

1. A medical intervention system for use during a medical intervention, comprising:
a needle guide comprising one or more orifices configured to guide needle insertion into a body for a medical intervention wherein the needle guide comprises restriction means for regulating the needle insertion resistance in the one or more orifices wherein the needle guide comprises an actuator configured for operating the restriction means in dependence of an input control signal and
a medical data unit configured for storing and/or acquiring medical data;
a control system, configured for receiving data from the medical data unit further configured to generate the input control signal for the restriction means based on the medical data in order to reduce the risk of penetration of a vulnerable area by the needle.

2. A medical intervention system as claimed in claim 1,
wherein the needle guide is a grid comprising a plurality of grid orifices arranged to assist in needle guidance during a medical intervention, wherein the plurality of grid orifices comprise individual restriction means and
wherein the medical intervention system further comprises a means for determination of the grid position with respect to a predetermined part of the body based on a position sensor or marker on the grid, or based on a calibration process;
wherein the medical data unit is configured for storing data comprising information on a grid orifice not to be currently used and wherein the control system is configured to generate the input control signal for the restriction means based on the current grid orifice not to be currently used and the grid position and wherein the restriction means are configured for blocking, stopping or restricting needle insertion through the grid orifice not to be currently used.

3. A medical intervention system as claimed in claim 1, wherein the medical data unit comprises
a medical imaging system, configured for image acquisition;
an image segmentation unit, configured for segmentation of a body structure in the image;
means for tracking a needle position, configured for tracking a position of the needle;
wherein the control system is configured to generate the input control signal for the restriction means based on a distance between the segmented body structure and the needle position.

4. A medical intervention system as claimed in claim 3, wherein the restriction means is configured for blocking, stopping or restricting needle insertion, when the needle tracking fails.

5. A medical intervention system as claimed in claim 3 configured to track the body structure in the image over time.

6. A medical intervention system as claimed in claim 5 wherein the restriction means is configured for blocking, stopping or restricting needle insertion, when the body structure tracking fails.

7. A medical intervention system as claimed in claim 1, wherein the needle guide comprises one or more orifices to assist in needle guidance during the needle insertion, wherein the restriction means are configured for sending an output signal to a control system, wherein the output signal comprises information if an orifice is open or closed by the restriction means; the system further comprising:
a control system configured for receiving the output signal from the restriction means and for determining what regions can be and/or cannot be reached from the one or more open orifices
a display configured for displaying the regions that can be and/or cannot be reached from the open orifices.
